(19) United States
(12) Patent Application Publication
(10) Pub. No.: US 2007/0203750 A1
(43) Pub. Date: Aug. 30, 2007

(54) METHOD AND APPARATUS FOR MANAGING HEALTH CARE INFORMATION

(76) Inventor: Julie Volcheck, Tallmadge, OH (US)

Correspondence Address: DAVID P DURESKA;BUCKINGHAM DOOLITTLE & BURROUGHS, LLP, 4518 FULTON DRIVE, NW, P O BOX 35548, CANTON, OH, 44735-5548, US

(21) Appl. No.: 11/678,150

(22) Filed: Feb. 23, 2007

Related U.S. Application Data
(60) Provisional application No. 60/776,292, filed on Feb. 24, 2006.

(51) Int. Cl. G06Q 10/00; A61B 5/00

(52) U.S. Cl. 705/2; 600/300

(57) ABSTRACT

A method for managing health care related information includes the steps of providing a computer database, and storing health care related information in the database A computer for use by a health care provider is provided, and the health care provider computer is operatively connected to the database. A plurality of health care management functions is displayed on the health care provider computers and the health care provider uses the health care provider computer to select a specific health care management function Information for the selected specific health care management function is retrieved from the computer database and is displayed on the health care provider computer The present invention also includes an apparatus for managing health care related information, including a computer network to carry out the steps of the method

### CROSS-REFERENCE TO RELATED APPLICATION

This application claims the benefit of U.S. Provisional Patent Application Ser. No. 60/776,292, which was filed on Feb. 24, 2006

### BACKGROUND OF THE INVENTION

1. Technical Field

The invention relates to the art of health care, and in particular to the management of information pertaining to health care More specifically, the invention is directed to a method and an apparatus for managing information pertaining to health care, such as insurance provider guidelines and office management documents, thereby enabling health care providers to reduce the amount of time spent on administrative tasks.

2. Background Art

Physicians and other health care providers must often undesirably devote time to administrative tasks, which reduces the amount of time the provider spends caring for patients. One of the primary administrative tasks for such providers is keeping current with guidelines and rules set forth by institutions that provide insurance coverage for medical services. These institutions include managed care institutions, insurance companies and government agencies, among others.

More particularly, the patients of health care providers often have some form of medical coverage or insurance to pay for at least a portion of certain medical services, such diagnoses and/or treatments Thus, rather than directly bill the patient for services rendered, the health care provider in such situations usually must bill the institution that provides the insurance. However, to be properly paid for the health care services that have been rendered, the provider must follow the rules established by each specific institution that provides the insurance for each respective patient Such rules include knowing and following certain policies of each insurance institution, such as a health care provider referral policy and a health care provider pre-certification policy, as well as which institutions participate in certain coverage plans.

Following these rules ensures that the health care provider will be reimbursed for services tendered at the highest agreed-upon level, and that the patient will receive the maximum benefit of his or her health care coverage. Failure to follow these rules may result in a denial by the insurance institution to pay for all or part of the services rendered by the health care provider, which causes the provider to forego payment, and/or causes the patient to be responsible for more expenses Of course, different insurance institutions establish different procedures, essentially creating a bureaucratic maze for the health care provider. In the prior art, there has been no central method or apparatus for keeping track and/or managing the rules, and changes to those rules, which are set forth by insurance institutions Rather, such information typically is released by a variety of different methods, depending on the particular institution For example, some institutions use newsletters, which may not be routed to the correct personnel associated with the health care provider to ensure proper tracking. Other institutions present such information at seminars, which are often not attended by many health care providers Still other institutions may include such information on their web sites, but since many of the web sites of the institutions are directed to patients Or employers of patients, the sites typically are difficult to navigate to get the information that is needed.

Moreover, while many institutions issue a manual containing the rules to the health care provider when the health care provider contracts with the insurance institution, the manuals are seldom updated to reflect changes in the rules. In addition, certain information that is helpful for health care providers, such as directories of specialists and health care facilities that are in the selected coverage network for each institution, typically are no longer printed and distributed to health care providers.

Thus, in many cases, information that is critical to the reimbursement of the health care provider by the insurance institution is unknown until a reimbursement claim is denied by the institution. For example, information regarding pre-certification of the health care provider with a specific insurance institution is often learned after a reimbursement claim has been denied, which typically prevents payment of the reimbursement claim by the institution The aforementioned difficulties of health care providers to keep current with the rules of insurance institutions become even more significant when they are coupled with the fact that such institutions are faced with continued rising costs in customer service to accommodate a large volume of calls regarding denied claims and pre-certification Therefore, it is desirable for health care providers to use automated technology) such as the Internet, to obtain pertinent insurance information, thereby increasing the efficiency of available resources.

In addition to administrative tasks associated with insurance institutions, health care providers must often devote time to other administrative tasks that are necessary to provide health care, such as office management. Such office management may include obtaining and completing health care related forms and the ordering of supplies These additional administrative tasks further undesirably reduce the amount of time a health care provider is able to spend with patients

As a result, a need exists in the art for a method and apparatus for obtaining and managing health care related information in a central system which is convenient for health care providers to use The method and apparatus of the present invention satisfy these needs.

### SUMMARY OF THE INVENTION

An objective of the present invention is to provide a method for obtaining and managing health care related information in a central system that is convenient for health care providers to use.

Another objective of the present invention is to provide an apparatus for obtaining and managing health care related information in a central system that is convenient for health care providers to use.

These objectives and advantages are obtained by the method for managing health care related information of the present invention In an exemplary embodiment of the invention, the method includes the steps of providing a computer database and storing health care related information in the database. A computer for use by a health care provider is provided, and the health care provider computer is operatively connected to the database A plurality of health care management functions is displayed on the health care provider computer; and the health care provider uses the health care provider computer to select a specific health care management function. Information for the selected specific health care management function is retrieved from the computer database and is displayed on the health care provider computer.

These objectives and advantages are also obtained by the apparatus for managing health care related information of the present invention. In an exemplary embodiment of the invention, the apparatus includes a computer network, which includes a computer database and health care related information that is stored in the database A computer server is operatively connected to the database, and a user computer for use by a health care provider is operatively connected to the computer server. The computer server accesses the computer database to retrieve selected health care related information for display on the user computer in response to a request from the health care provider on the user computer.

### BRIEF DESCRIPTION OF THE DRAWINGS

The preferred embodiments of the present invention, illustrative of the best modes in which applicant has contemplated applying the principles, are set forth in the following description and are shown in the drawings, and are particularly and distinctly pointed out and set forth in the appended claims.

FIG. 1 is a flowchart showing general steps of the method of the present invention;

FIG. 2 is a flowchart showing detailed steps of the insurance summary function portion of the method shown in FIG. 1;

FIG. 3 is a flowchart showing detailed steps of the pharmacy information function portion of the method shown in FIG. 1;

FIG. 4 is a flowchart showing detailed steps of the formulary information function portion of the method shown in FIG. 1;

FIG. 5 is a flowchart showing detailed steps of the coding information function portion of the method shown in FIG. 1;

FIG. 6 is a flowchart showing detailed steps of the hospital information function portion of the method shown in FIG. 1;

FIG. 7 is a flowchart showing detailed steps of the office management function portion of the method shown in FIG. 1;

FIG. 8 is a flowchart showing detailed steps of the office supplies function portion of the method shown in FIG. 1;

FIG. 9 is a flowchart showing detailed steps of the insurance provider directories function portion of the method shown in FIG. 1;

FIG. 10 is a flowchart showing detailed steps of the fee information function portion of the method shown in FIG. 1;

FIG. 11 is a flowchart showing detailed steps of the program outline information function portion of the method shown in FIG. 1;

FIG. 12 is a flowchart showing detailed steps of the disease management function portion of the method shown in FIG. 1;

FIG. 13 is a flowchart showing detailed steps of the diagnosis information function portion of the method shown in FIG. 1;

FIG. 14 is a flowchart showing detailed steps of the news information function portion of the method shown in FIG. 1;

FIG. 15 is a flowchart showing detailed steps of the reports function portion of the method shown in FIG. 1;

FIG. 16 is a flowchart showing detailed steps of the electronic remittance function portion of the method shown in FIG. 1;

FIG. 17 is a flowchart showing detailed steps of the practice management function portion of the method shown in FIG. 1;

FIG. 18 is a flowchart showing detailed steps of the patient eligibility function portion of the method shown in FIG. 1;

FIG. 19 is a flowchart showing detailed steps of the point of care payment information function portion of the method shown in FIG. 1;

FIG. 20 is a flowchart showing detailed steps of the claim information function portion of the method shown in FIG. 1; and

FIG. 21 is a schematic block diagram showing selected components of an exemplary apparatus of the present invention.

Similar numerals refer to similar parts throughout the drawings.

### DESCRIPTION OF THE PREFERRED EMBODIMENTS

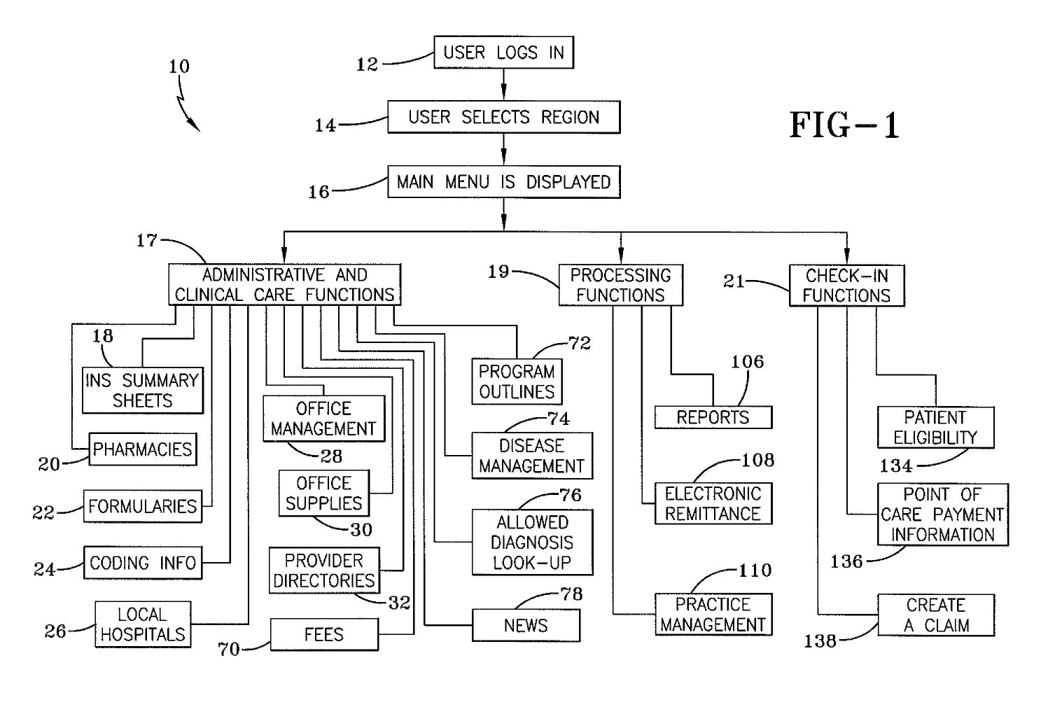

With reference now to FIG. 1, an exemplary embodiment of the method for managing health care information of the present invention is shown and is indicated generally at 10. Method for managing health care information 10 includes obtaining information from insurance institutions, such as the rules and guidelines of each institution and any updates thereto, and centrally storing the same for easy and convenient access by health care providers Conversely, method for managing health care information 10 provides a means for insurance institutions to disseminate such information in a cost efficient and effective manner. Moreover, method for managing health care information 10 includes a method for storing and retrieving other information that is useful for health care providers, such as frequently used forms.

As will be described in greater detail below, method for managing health care information 10 preferably utilizes an electronic network to access information contained in a central location, such as by using a computers that is connected to the Internet, which operably connects to a server that accesses a database of health care information. To access desired health care information, a health care provider uses the computer to connect to the Internet and accesses a specific web site, step 12 The web site may optionally be a secure site, requiring the health care provider to enter a user name and/or password of other specific information to access or log into the web site, as known in the art.

Once the health care provider accesses the web site, the health care provider preferably selects his or her geographic region from a display of icons that represent geographic regions, step 14. As referred to herein, the term “select” is used to denote electronic selection on a computer in which a user employs a mouse or other device to click on an icon or electronic link, as known in the computer art Such geographic selection is preferable since many rules and guidelines of insurance institutions vary by state or other geographic regions, and selection of a specific region enables access to the proper information for that region. Once the proper legion is selected, a main menu is displayed, step 16, which enables the health care provider to view and select icons that corresponds to convenient groupings of specific health care information management functions. For example, such groupings of management functions may include, administrative and clinical care functions 17; processing functions 19; and check-in functions 21

Each grouping 17, 19, 21 in turn includes specific management functions For example, administrative and clinical care functions 17 preferably include: insurance summary sheets 18; pharmacy information 20; formulary information 22; coding information 24; local hospital information 26; office management information 28; office supply information 30; insurance institution directory information 32; fee information 70; program outlines 72; disease management information 74; diagnosis look-up information 76; and news 78 Processing functions 19 preferably include: reports 106; electronic remittance 108; and practice management 110. Check-in functions 21 preferably include: patient eligibility 134; point of care payment information 136; and creation of a claim 138.

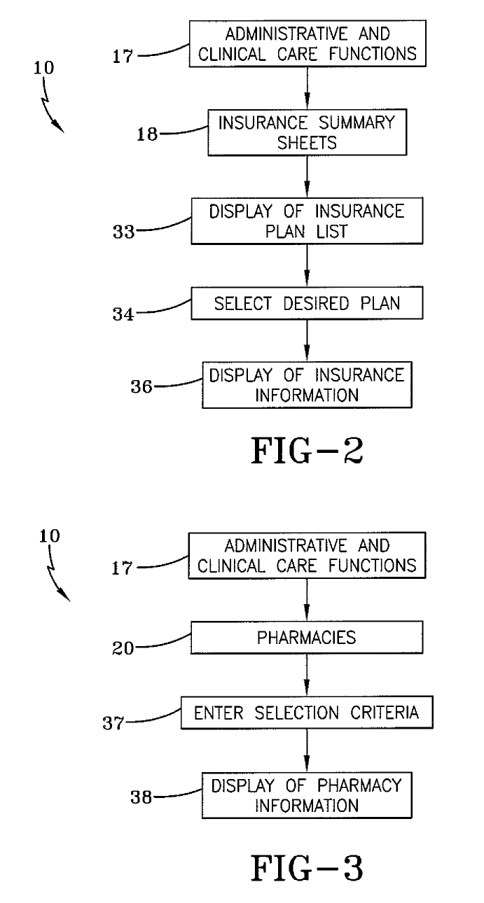

With additional reference now to FIG. 2, when a health care provider accesses administrative and clinical care functions 17 from main menu in step 16, and chooses insurance summary sheets 18, information on specific insurance plans may be obtained For example, in step 33, a list of insurance plan summaries is displayed, preferably in predetermined categories such as National Plans, Government Plans, Regional Plans and Behavioral Health Plans The health care provider then selects a desired plan from the list, step 34 Once a desired plan is selected, a summary of the selected plan is displayed, with links that enable the health care provider to access screens containing more detailed information, step 36 For example, the display may include a description of the insurance plan and contact information for the insurance institution, such as a phone number and mailing address. The links to more detailed information may include links to screens containing the rules of the institution for health care provider referral, pie-certification requirements, and directories of participating physical therapists, hospitals, laboratories, urgent care centers, durable medical equipment (DME) suppliers and/or healthy outlook programs sponsored by or affiliated with the institution.

The institutional information accessed in step 36 may be provided directly by the institution, or by a facilitator of the web site, as will be described in greater detail below In this manner, the information will reflect the current rules and practices of the insurance institution, and is easily accessible by a health care provider. Method of managing health care information 10 therefore standardizes the conveyance of insurance information from insurance institutions to health care providers such as physicians, hospitals, and healthcare staff. Moreover, method of managing health care information 10 uses the technology of the Internet to provide insurance information in a standardized, template form accessible in a single location

With reference now to FIG. 3, when a health care provider chooses pharmacy information function 20, the provider is easily able to locate a local pharmacy Mote particularly, health care providers must of ten telephone a certain pharmacy to place a prescription for a patient. In the prior art, this involved asking the patient for the pharmacy phone number, keeping a separate list of pharmacies and their phone numbers, or looking the phone number up in a telephone book. Method of managing health care information 10 enables a health care provider to select pharmacy function 20, which in turn enables the provider to enter selection criteria such as a zip code, step 37, and displays names and telephone numbers of pharmacies within the specified selection criteria, step 38. In this manner, a single central system also provides pharmacy contact information for health care providers.

Turning now to FIG. 4, selection of formulary information function 22 generates a list of formularies, or medications that certain insurance institutions will pay for. Since formularies are updated of ten by insurance institutions, current information from each institution is important for the health care provider and the patient. Thus, in step 39, a list of insurance institutions preferably is displayed, which enables a health care provider to select the name of a specific patient's insurance institution in step 40, which leads to a display of specific coverage plans offered by that institution and formularies for each plan in step 41 The formulary information displayed in step 41 may be provided directly by the institution, or by a facilitator of the web site, as will be described in greater detail below In this manner; the formulary information will likely be current and is easily accessible by a health care provider.

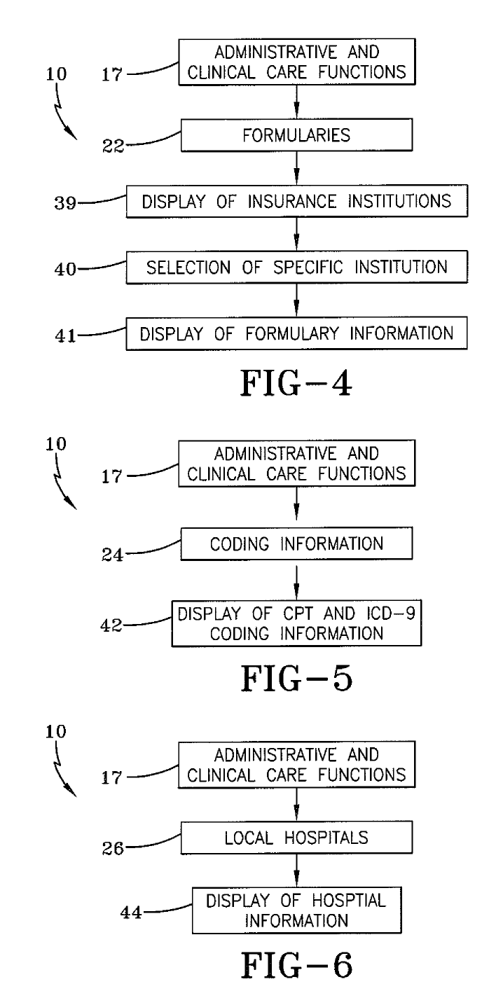

With reference now to FIG. 5, selection of coding information function 24 enables the health care provider to remain current on Current Procedural Terminology, which is known in the art as CPT, and on the International Classification of Disease, which is known in the art as ICD-9 CPT is developed and published by the American Medical Association (AMA), and is a listing of descriptive terms and identifying codes for reporting medical services and procedures The Administrative Simplification Section of the Health Insurance Portability and Accountability Act of 1996 (HIPAA) names CPT as the procedure code set for physicians to submit claims to insurance institutions for reimbursement Since the AMA deletes, adds and revises the CPT on an on-going basis, it is necessary for a health care provider, such as a physician, to be informed of the changes to ensure that the health care provider's coding of claims is accurate, thereby enabling correct processing by the insurance institution for reimbursement.

ICD-9 is used to assign a specific code to each known disease or condition, and the codes are in turn listed on an insurance claim form to report a physician's diagnosis of a patient TCD-9 is jointly maintained by the National Center for Health Statistics and the Center for Medicare and Medicaid Services. Insurance institutions of ten specify which tests are covered for certain diseases of conditions by referencing the ICD-9 code Like the CPT, revisions, deletions and additions are made to the ICD-9 on an on-going basis, and health care providers must be kept informed of these changes to ensure proper processing and reimbursement of their claims by insurance institutions.

Thus, in step 42, icons enabling a user to access and view CPT and ICD-9 codes are displayed, which enables the health care provider to select and view the appropriate codes and the rules for coverage that correspond to each code, as set forth by each insurance institution.

Turning now to FIG. 6, selection of local hospital information function 26 enables the health care provider to obtain convenient access to information on local area hospitals. Since health care providers of ten need to contact local hospitals, or verify certain information about such hospitals, it is convenient to have the hospital information located in a central system Since the health care provider specifies his or her geographic region in step 14 above, when local hospital function 26 is selected, local hospital information is displayed, step 44 Such information may include contact information, such as a phone number and address for each hospital, hospital policy information, maps to each hospital, and the like.

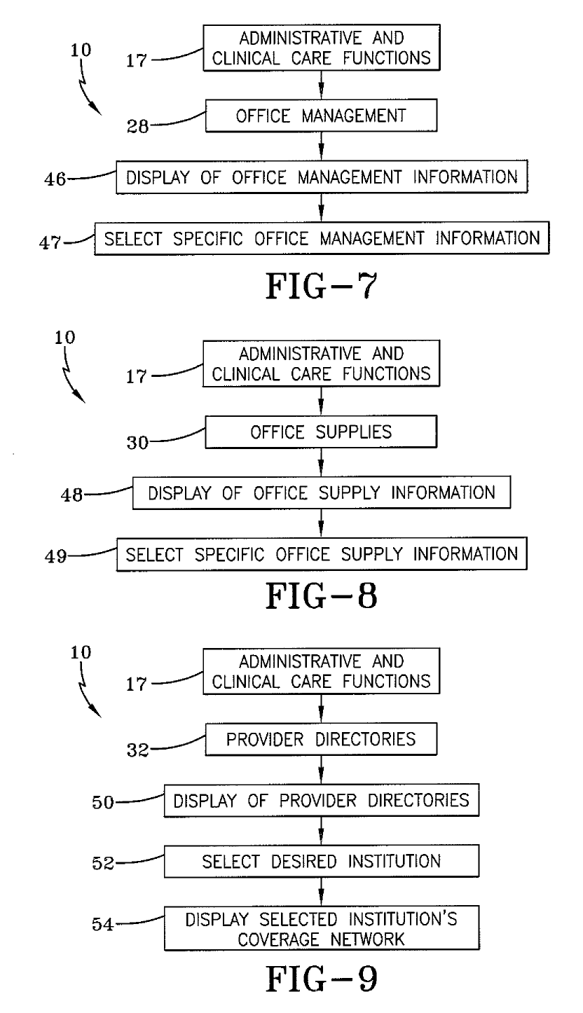

With reference now to FIG. 7, when the health care provider selects the office management information function 28, office information is obtained. More particularly, in step 46, icons are displayed that list specific health care provider office information, such as information for the credentialing of the health care provider, general office forms, forms for the Health Insurance Portability and Accountability Act of 1996, Center for Disease Control (CDC) vaccine schedules, and the like. In step 47, the health care provider is able to select the specific office information that is desired, which then leads to the appropriate forms and instructions related to the selected information In this manner, method for managing health care information 10 also provides a central system for coordinating forms, pre-certification, and other office information.

Turning now to FIG. 8, when the health care provider selects the office supply information function 30, icons are displayed that represent sources for office supplies, step 48. Since many office supply companies enable users such as health care provider's to directly access their respective web sites to order supplies, it is easy and convenient for the health care provider to select an appropriate icon for a corresponding supply company, step 49, and thus access the web site of the selected supply company to order office supplies using the central system provided by method for managing health care information 10.

With reference now to FIG. 9, when the health care provider selects the insurance institution directory information function 32, also known as the insurance provider directories function, the provider is able to obtain information regarding other health care providers that are part of a specific insurance institution's coverage network. Often, a health care provider must find other providers that participate in a certain insurance institution's coverage plan However, it is necessary in the prior art to separately visit the specific web site for each insurance institution, rather than accessing a central system. In step 50, a list of insurance institutions is displayed, and the name of the desired institution is selected in step 52, which leads to a display or listing of which health care providers are included in that institution's coverage network in step 54. Since this information may be provided directly by the institution, or by a facilitator of the web site, the directory will reflect the names and contact information of health care providers that ate currently in each institution's respective coverage network.

Turning now to FIG. 10, selection office information function 70 enables a health care provider to access specific information regarding reimbursement of the health care provider by an insurance institution. More particularly, many patients have health care benefits with a deductible or coinsurance that is based on an amount which is allowed by the insurance institution or is contracted between the insurance institution and the health care provider. Thus, the contract between the health care provider and each insurance institution typically prohibits the health care provider from collecting fees that exceed an amount that is specified in the contract In order to determine the proper amount offer that the health care provider should collect from the patient at the time of service, the health care provider needs a tool to calculate that amount

Method for managing health care information 10 provides such a calculation tool. When the fee information function 70 is selected, insurance plans and reimbursement selection information is displayed. Preferably, the reimbursement selection information is based upon CPT codes, which awe described above Thus, the health care provider selects the patient's insurance plan and the CPT code for the service that is being provided, step 80, and the particular reimbursement information for the CPT code that was selected is displayed, step 82 The health care provider is then able to collect the proper fee from the patient at the time the service is rendered.

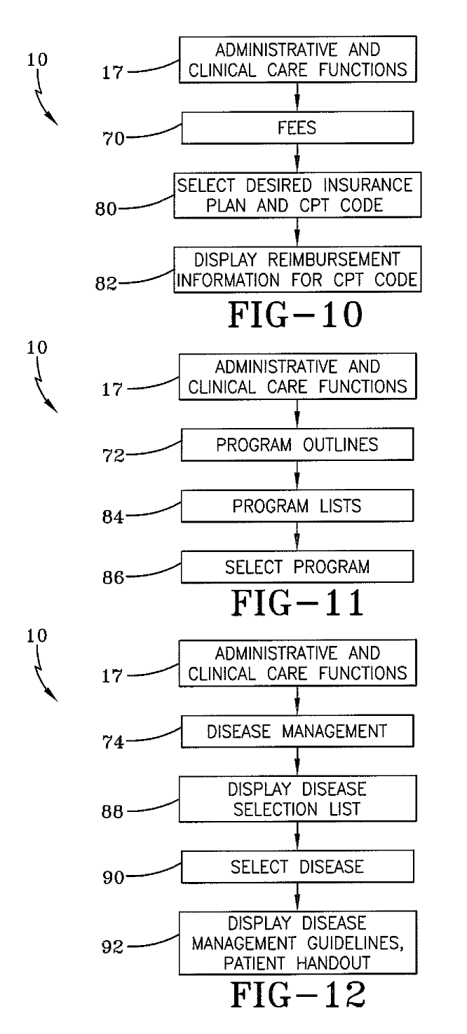

With reference now to FIG. 11, selection of program outlines function 72, enables the health care provider to review information on how to establish health and wellness programs for patients by organizing the provider's resources, such as the provider's staff and documentation. Such programs help to promote and improve the overall health of a patient, and may be reimbursable by an insurance institution using the above-described CPT guidelines. Thus, in step 84, lists of health and wellness programs are displayed. The health care provider selects a desired program in step 86, and is able to review information related to the selected program, such as insurance coverage criteria, relevant CPT codes, insurance reimbursement information, related health care provider forms, and resources for additional reference materials and training.

Turning now to FIG. 12, when the health care provider selects disease management information function 74, the provider receives information and resources to develop the provider's practice policy and procedure for managing diseases. More particularly, it is desirable to provide a tool to health care providers that gives the providers the information to set policy and procedures for managing patients with chronic diseases, as well as to ensure that patients receive preventive services and proper, proactive care to keep the disease well-controlled, which keeps the patient healthier. Such information will desirably control health care costs, as hospital admissions will be reduced since patients will be healthier. Thus, in step 88 a list of diseases to be managed is displayed, and the health care provider selects the disease to be managed in step 90. In step 92, information related to management of the selected disease is displayed, including disease management guidelines, such as frequency of visits to the health care provider; laboratory tests and procedures, screening guidelines and criteria, and benchmarks and goals Also preferably displayed in step 92 is a handout for patients with information regarding the disease and its management, which can be printed out and given to the patient by the health care provider.

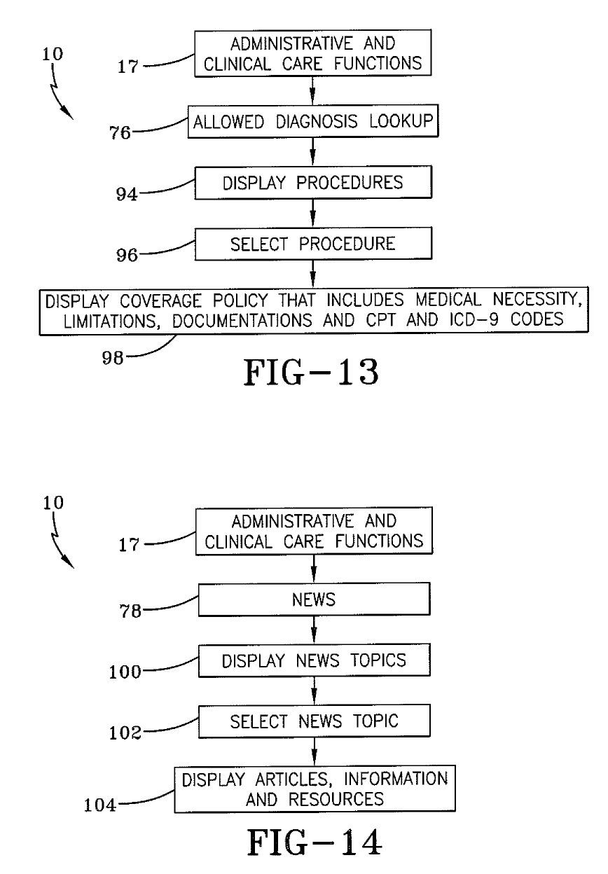

With reference now to FIG. 13, selection of allowed diagnosis look-up information function 76 enables the health care provider to access insurance institution rules for coverage of tests and procedures for certain diseases or conditions. More particularly, as described above, CPT codes are assigned to specific procedures and tests, and ICD-9 codes are used to assign a specific code to each known disease or condition. Insurance institutions of ten specify which procedures ate covered by referencing the CPT code, and/or which tests are covered for certain diseases or conditions by referencing ICD-9 code. Thus, in step 94, a list of procedures is displayed, enabling a user to select a specific procedure in step 96. In step 98, information on the specific procedure is displayed, such as CPT codes, ICD-9 codes, and rules set forth by each insurance institution for coverage of tests that correspond to each code, preferably including medical necessity of the tests, limitations of coverage and related documentation.

Turning now to FIG. 14, selection of news function 78 enables the health care provider to review a list of current topics and regulations set forth by insurance institutions, including Medicare and Medicaid Since health care providers typically have contracts with insurance institutions, and each insurance institution has its own defined rules and networks, it is desirable to keep the health care provider informed of such topics and regulations, which enables the provider to deliver services and manage the administration of services in conformance with the contract between the provider and each respective insurance institution. In step 100, news regarding topics and regulations is displayed, and the health care provider then selects a desired news topic or regulation, step 102. Specific information on the selected news topic or regulation, such as articles and resources, is displayed in step 104. In this manner; method for managing health care information 10 provides a central system that enables health care provider's to access updates by insurance institutions, which reduces errors and associated costs in processing insurance information

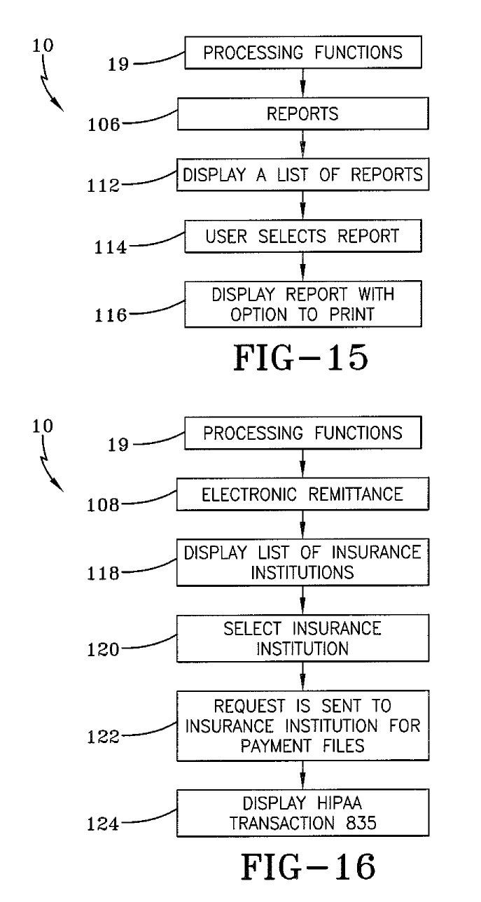

With reference now to FIG. 15, when a health care provider accesses processing functions 19 and selects reports function 106, the health care provider is able to receive deposits regarding claims and transactions. In step 112, a list of report types is displayed, such as reports of claims that have been sent to insurance institutions, reports on patients, and reports on transactions such as services provided for a given range of time. The desired report is selected by health care provider in step 114, and the selected report is displayed in step 116. For convenience, step 114 also preferably includes an option to print the selected report for review by the health care provider

Turning now to FIG. 16, selection of electronic remittance function 108 enables the health care provider to receive an electronic remittance file from the insurance institution An electronic transaction from an insurance institution to a health care provider is known to those skilled in the health care provider art as a HIPAA transaction 835. A HIPAA transaction 835 typically is used by an insurance institution to send a reimbursement or payment for services to a health care provider who has submitted a claim, and/or to send an explanation of payment information to the health care provider Such electronic remittance files typically enable the health care provider to receive a faster response to claims from an insurance institution than if submitted by regular mail In addition, such electronic remittance files provide a format that the health care provider can archive for future reference, and which can be used in interfacing with the accounting system of the health care provider. Thus, when electronic remittance is selected in step 108, a list of insurance institutions is displayed in step 118, and the health care provider selects a desired insurance institution, step 120. The health care provider then is able to send a request for payment files to the insurance institution, step 122, and the HIPAA transaction 835 is displayed in step 124 for the health care provider to download and/or print.

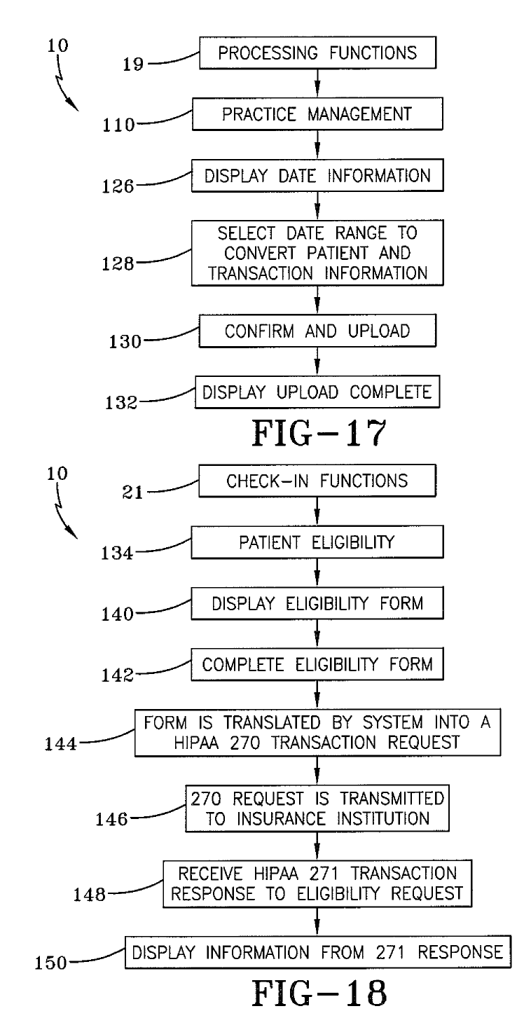

With reference now to FIG. 17, selection of practice management function 110 enables the health care provider to conveniently upload and coordinate patient transaction files for accounting purposes. More particularly, as will be described in greater detail below, specific identification information for each patient is gathered and saved using patient eligibility function 134, while payment and transactional information for each patient is gathered and saved using point of care payment information function 136. It is of ten beneficial to a health care provider to coordinate this information to create an accounts receivable file or ledger for patients, which shows the date of service and the procedures provided. Since all of this information is centrally located through method for managing health care information 10, when practice management function 110 is selected, date information and patient identification information is displayed, step 126. The health care provider selects the date range of services and identifies the patient, step 128. The files are retrieved and uploaded, step 130, and are displayed for the provider, 132

Turning now to FIG. 18, when a health care provider accesses check-in functions 21 and selects patient eligibility function 134, a real-time request is able to be created to verify a patient's insurance coverage. As described above, insurance coverage for patients is an important consideration for any health care practitioner. By knowing what coverage a patient has before services are provided, the health care provider is informed of the guidelines and rules set forth by the patient's insurance institution regarding delivery of 'services and care of the patient. In addition, should a patient's coverage be expired or otherwise limited or nonexistent, the health care provider can be informed of this and address it with the patient at the time of service, thus reducing costs and errors in submitting claims to insurance institutions

In step 140, a patient eligibility form is displayed, which the health care provider completes electronically by filling in data fields with the name, address and insurance information of the patient, step 142 An electronic transaction from a health care provider to an insurance institution to inquire about the health care coverage associated with a particular patient is known to those skilled in the health care provider art as a HIPAA 270 transaction request. In step 144 the information entered by the health care provider is translated into a HIPAA 270 transaction request, which is then transmitted to the insurance institution in step 146. The response of the insurance institution to the HIPAA 270 transaction request is known in the art as a HIPAA 271 transaction response, and is sent to the health care provider. In step 148, method for managing health care information 10 receives the HIPAA 271 transaction response, which verifies the patient's insurance coverage and lists specific information regarding the patient's coverage, such as copay amounts, deductible amounts, and preventive services. This information is displayed for the health care provider, step 150.

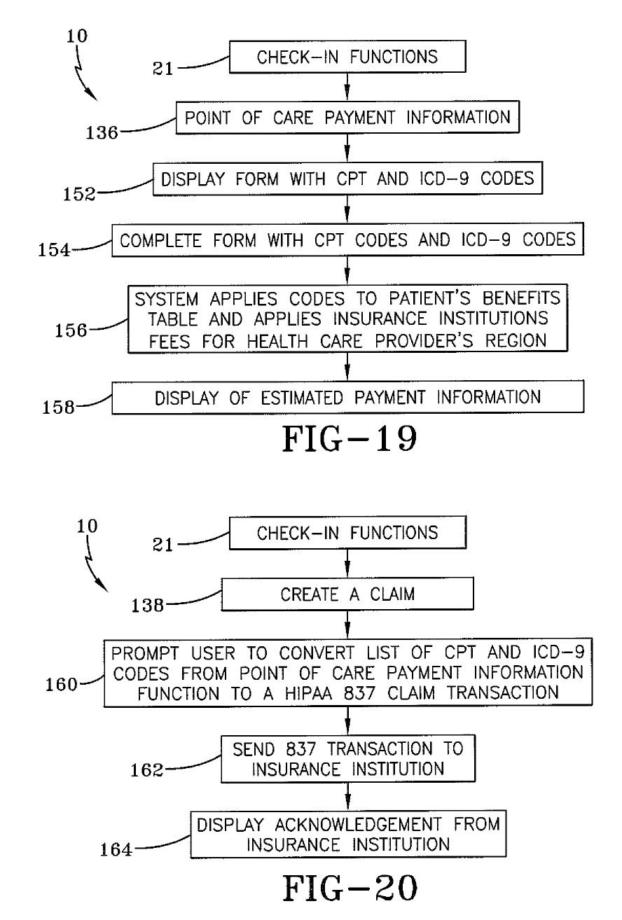

With reference now to FIG. 19, selection of point of care payment information function 136 enables a health care provider to calculate what portion of services a patient's insurance institution will reimburse, which allows the provider to collect the balance from the patient at the time the services are provided. More particularly, health care providers typically must submit a claim for reimbursement to a patient's health care institution after the services have been provided, and after receiving a response from the insurance institution regarding reimbursement, the providers then send a bill to the patient for any amount that has not been reimbursed by the insurance institution. This process creates a significant undesirable delay for the health care provider between the time of the rendering of services and full payment, and sometimes may result in less than full payment. By calculating at the time the services are provided the amount the patient's insurance institution will reimburse, the health care provider is able to collect payment at the point of care and thus receive prompt full payment.

As described above, insurance institution reimbursement information of ten corresponds to CPT and ICD-9 codes Thus, in step 152, a form with CPT and ICD-9 codes for the patient is displayed, which is completed by the health care provider in step 154 The CPT and/or ICD-9 codes that have been entered are then applied to the fees of the insurance institution of the patient in step 156. Estimated payment information, preferably including the amount of reimbursement by the patient's insurance institution and any amount for which the patient is responsible to pay, is displayed for the health care provider in step 158 In this manner method for managing health care information 10 enables the health care provider to calculate and collect at the point of care the proper amount owed by the patient

Turning now to FIG. 20, selection of create a claim function 138 enables the health care provider to conveniently prepare and send an electronic file for a reimbursement claim to an insurance institution More particularly, an electronic filing of a claim by a health care provider for reimbursement by an insurance institution is known in the art as a HIPAA 837 transaction. The health care provider is required by insurance institutions to send a HIPAA 837 transaction to the institutions in order to receive payment for services provided to the insured patient. Rather than re-entering information to generate the HIPAA 837 transaction, method for managing health care information 10 is able to take previously entered information and generate the transaction.

As described above, insurance institution reimbursement information of ten corresponds to CPT and ICD-9 codes, which have been entered in point of care payment information function 136 for the particular services rendered by the health care provider When create a claim function 138 is selected, the user is prompted in step 160 to convert the list of CPT and ICD-9 codes entered in point of care payment information function 136 into a HIPAA 837 transaction. The prepared HIPAA 837 transaction is sent to the insurance institution, step 162, and an acknowledgement from the insurance institution of receipt of the HIPAA 837 transaction from the health care provider is displayed, step 164

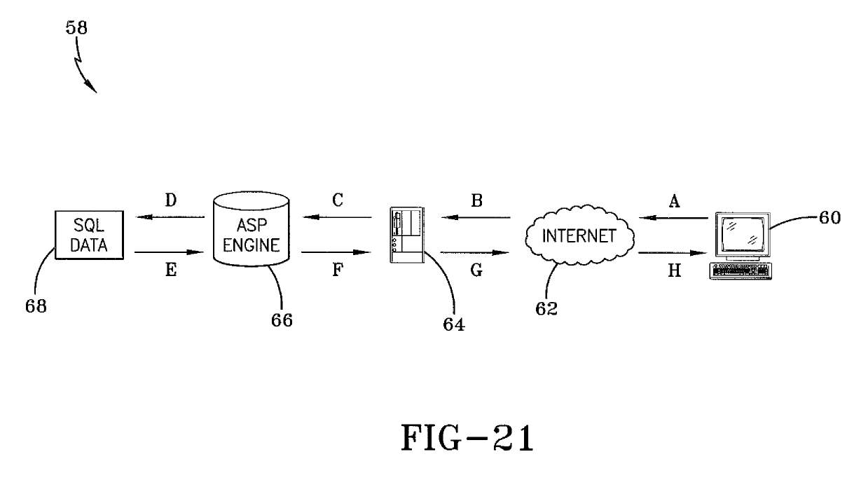

Turning now to FIG. 21, the apparatus for managing health care information according to method 10 is shown and is indicated generally at 58. Apparatus 58 utilizes a computer network to access information contained in a central location For example, a user computer 60 for a health care provider is operably connected to a central database 68 of health care information. The connection preferably is via an inter-network of computers, and more preferably via Internet 62

More particularly, a health care provider uses computer 60 to access Internet 62 as known in the art, Arrow A, and enters the domain name of a uniform resource locator (URL) on the Internet, Arrow B, which corresponds to a server 64. Server 64 is a computer that supplies information pursuant to a request from the health care provider's computer 60 Server 64 accesses database 68, Arrows C and D, preferably via an application service provider (ASP) engine 66, which is a third-party software distribution and/or management service that provides software via a wide area network from a centralized data center.

Database 68 is a large collection of data organized for rapid search and retrieval, and includes the above-described information for the health care provider. Preferably, database 68 is a structured query language (SQL) database, which is a database that uses the SQL computer language to store, manipulate, and retrieve the data that is stored in the database. The above-described information for the health care provider preferably is organized and storied in the form of data tables, as known in the computer programming art, which facilitates rapid storage and retrieval of the data. The specific information requested by the health care provider is thus retrieved from database 68 and returns to the provider's computer 60 as denoted by Arrows E, F, G and H, for display on the computer.

Thus, apparatus 58 enables the execution of method for managing health care information 10. More particularly, the health care provider uses computer 60 to access the Internet 62 and logs into the website for server 64, as described above in step 12 of the method (FIG. 1). The health care provider then selects a desired function and receives the information relating to that function, as facilitated by server 64 accessing ASP engine 66 and database 68

It should be noted that, while reference above has been made to access by a health care provider, web server 64 may also be accessed via the Internet by insurance institutions to disseminate rules and other information, thereby enabling the information in database 68 to remain current Alternatively, a facilitator of web server 64 and/or database 68 may obtain rules and other information from insurance institutions and update the information in the database accordingly

Thus, the present invention provides a method and an apparatus that incorporate world-wide-web technology and display, via the Internet, information relating to the administration of health care plans in a straightforward, easy-to-use interface. The method and apparatus for managing health care information of the present invention streamline the work flow of the health care provider and reduce the amount of paper generated in the health care provider's office in the administration of managed care plans.

Moreover, the method and apparatus for managing health care information of the present invention reduce the administrative complexity of managed care and government health plans, such as Medicate and Medicaid, by providing physicians and other health care providers with a significant reduction in overhead to administer the plans, resulting in greater focus on patient care. The method and apparatus of the present invention also improve the ability of the health care piovider's staff to obtain the correct prior authorization from insurance institutions before performing procedures and tests.

In this manner, the method and apparatus of the present invention obtain and manage health care related information in a central system which is convenient for health care providers to use. It should be noted that the steps, groupings and functions of the above-described method may be altered, for certain steps, groupings and/or functions omitted, without affecting the concept or operation of the invention.

In the foregoing description, certain terms have been used for brevity, clarity and understanding; but no unnecessary limitations are to be implied therefrom beyond the requirements of the prior art, because such terms ate used for descriptive purposes and are intended to be broadly construed. Moreover, the present invention has been described with reference to exemplary embodiments. It shall be understood that this illustration is by way of example and not by way of limitation. Potential modifications and alterations will occur to others upon a reading and understanding of this disclosure, and it is understood that the invention includes all such modifications and alterations and equivalents thereof.

Accordingly, the method and apparatus for managing health care information of the present invention are simplified, provide an effective, safe, inexpensive and efficient method and structure which achieve all of the enumerated objectives, provide for eliminating difficulties encountered with prior art methods and apparatus for managing health care information, and solve problems and obtain new results in the art.

Having now described the features, discoveries and principles of the invention, and the manner in which the improved method and apparatus for managing health care information are constructed, arranged and used, as well as the characteristics of the construction and arrangement, and the advantageous, new and useful results obtained, the new and useful structures, devices, elements, arrangements, parts, combinations and methods are set forth in the appended claims.

What is claimed is:

1. A method for managing health care related information, said method comprising the steps of:
providing a computer database;
storing health care related information in said database;
providing a computer for use by a health care provider;
operatively connecting said health care provider computer to said database;
displaying a plurality of health care management functions on said health care provider computer;
enabling the selection of a specific health care management function by said health care provider using said health care provider computer;
retrieving information for said selected specific health care management function from said computer database; and
displaying information for said selected specific health care management function on said health care provider computer.

2. The method for managing health care related information of claim 1, wherein said operative connection of said health care provider computer to said database is through the Internet.

3. The method for managing health care related information of claim 1, further comprising the step of enabling said health care provider to select a geographic region to display said plurality of health care management functions on said health care provider computer.

4. The method for managing health care related information of claim 1, wherein at least one of said plurality of health care management functions includes insurance plan information.

5. The method for managing health care related information of claim 1, wherein at least one of said plurality of health care management functions includes pharmacy information.

6. The method for managing health care related information of claim 1, wherein at least one of said plurality of health care management functions includes formulary information.

7. The method for managing health care related information of claim 1, wherein at least one of said plurality of health care management functions includes coding information for procedural terminology and diagnosis

8. The method for managing health care related information of claim 1, wherein at least one of said plurality of health care management functions includes local hospital contact information.

9. The method for managing health care related information of claim 1, wherein at least one of said plurality of health care management functions includes health care provider office management information.

10. The method for managing health care related information of claim 9, wherein said health care provider office management information includes at least one of information for credentialing, office forms, forms for the Health Insurance Portability and Accountability Act of 1996, and vaccine schedules.

11. The method for managing health care related information of claim 1, wherein at least one of said plurality of health care management functions includes health care provider office supply information.

12. The method for managing health care related information of claim 1, wherein at least one of 'said plurality of health care management functions includes information on health care providers that are part of a health care provider network of a selected insurance institution

13. The method for managing health care related information of claim 1, wherein at least one of said plurality of health care management functions includes fee information.

14. The method for managing health care related information of claim 1, wherein at least one of said plurality of health care management functions includes information on health and wellness program outlines.

15. The method for managing health care related information of claim 1, wherein at least one of said plurality of health care management functions includes disease management information.

16. The method for managing health care related information of claim 1, wherein at least one of said plurality of health care management functions includes information on insurance institution rules for tests and procedures for specified diseases.

17. The method for managing health care related information of claim 1, wherein at least one of said plurality of health care management functions includes news from insurance institutions

18. The method for managing health care related information of claim 1, wherein at least one of said plurality of health care management functions includes reports of claims and transactions

19. The method for managing health care related information of claim 1, wherein at least one of said plurality of health care management functions includes electronic claim remittance to an insurance institution.

20. The method for managing health care related information of claim 1, wherein at least one of said plurality of health care management functions includes information on accounts receivable practice management

21. The method for managing health care related information of claim 1, wherein at least one of said plurality of health care management functions includes verification of patient insurance information.

22. The method for managing health care related information of claim 1, wherein at least one of said plurality of health care management functions includes information on point of care payment calculations.

23. The method for managing health care related information of claim 1, wherein at least one of said plurality of health care management functions includes information for the creation of an electronic claim form.

24. A computer network for managing health care related information according to the method of claim 1

25. An apparatus for managing health care related information, said apparatus comprising:
a computer network, including:
a computer database;
health care related information stored in said database;
a computer server operatively connected to said database; and
a user computer for use by a health care provider, said user computer being operatively connected to said computer service; and
whereby said computer server accesses said computer database to retrieve selected health care related information for display on said user computer in response to a request from said health care provider on said user computer.

26. The apparatus for managing health care related information of claim 25, wherein said database is a structured query language database.

27. The apparatus for managing health care related information of claim 25, wherein said health care related information is stored in said database in data tables.
